(19) United States
(12) Patent Application Publication
(10) Pub. No.: US 2011/0106032 A1
(43) Pub. Date: May 5, 2011

(54) DEVICE FOR A TEMPORARY ILEOSTOMY OR UROSTOMY CLOSURE

(76) Inventor: Peter Kratky, Starnberg (DE)

(21) Appl. No.: 12/809,648

(22) PCT Filed: Dec. 19, 2008
(86) PCT No.: PCT/EP08/10907

§ 371 Date: Jan. 14, 2011

(30) Foreign Application Priority Data
Dec. 21, 2007 (DE) 10 2007 062 133.9

(51) Int. Cl. A61F 5/445

(52) U.S. Cl. 604/337

(57) ABSTRACT

The invention relates to a temporary seal for a stoma, which seal is designed in such way that fluid emerging during exchange of the seal is received in a receptacle or, when inserted into the intestinal lumen, already receives fluid there in a receptacle. The device for temporary closure of a stoma comprises a base plate 105 for permanent application in the periphery of a stoma on the outer face of an abdominal wall 2, and a seal which is connected releasably to the base plate 105 to form a temporary stoma closure and is configured in such a way that it forms a receiving volume for receiving at least on receptacle for fluid emerging from the stoma 3.

### FIELD OF THE INVENTION

The present invention relates to a device for temporary seal of a stoma, e.g. ileostoma or urostoma or colostoma.

### BACKGROUND OF THE INVENTION

A device of the type mentioned above is used in persons with a surgically made opening in a hollow organ to the body surface, eg an artificial anus, called a stoma. The word stoma derives from Greek and means “mouth” or “opening”. Appropriate prefixes the term stoma are divided into various intestinal outputs. One speaks of exit: of the small intestine of an aleostomy and of the colon of a colostomy and the diversion of urine of an urostoma. The colostoma is again divided into descendostoma or sigmoidostoma and transversostoma, the urostoma is divided between a uretherfistula to the skin and a conduit.

A stoma can be created temporary or for a long time (permanent). A stoma can be single lumen (terminally) or dual lumen (double-barreled).

The most common reason for the construction of a stoma is a cancer of the rectum (Rektumcarcinom) or of the anus (Analcarcinom). During the surgical removal of the tumor, it may be necessary also to remove the intestinal sphincter, and an artificial anus must be constructed on the belly.

For some surgical procedures, the natural sphincter may be preserved in some circumstances. In this case only a temporary colostoma is created to ensure the healing of the interface between the large intestine (Anastomosisprotection).

The second most common cause of the disease is a chronic inflammatory bowel disease such as Crohn's disease or Colitis ulcerosa. These diseases can be obtained by a stoma bowel, for example, are lit, lay still for a certain period or permanently. Do you have to remove the diseased intestine partly or wholly, a stoma may also be necessary.

With a colostoma (anus praeter) in the ileum of liquid, aggressive gut contents are emptied through the stoma. The amount of liquid, depending on the drinking and eating habits at about 2,500 ml/day. One distinguish a terminal and double-barreled ileostomy. What type is created depends on the disease.

With a colostoma (anus praeter) of the large intestine are more or less formed stool discharged through the colostomy. The gas formation is large in colostomy.

As for the distinction is a terminal ileostomy and a double-colostomy: A terminal stoma system is created every time is a disease in the rectum or the anus, depending on the condition rectum and anus are removed with a sphincter. In this case, the stoma is permanent. Other diseases require a temporary closure of the affected bowel section, so that can be transferred back to the healing of the stoma again. In a urostoma the stoma is to create diversion of urine. Due to various diseases of the urinary system, there is a loss of its function. To ensure this again, proven methods of operation are necessary, which usually lead to the artificial urinary diversion. The most common forms of urinary diversion are the Ureterfistula (Ureter-skin-fistula), the Ileum-Conduit, the Colon-Conduit or in rare cases the Uretherfistula.

There are dry and wet Urostomata. In the wet stoma as in the ureter-skin fistula, the deposed from the bladder ureter is drawn directly through the abdominal wall and attached to the skin. The ureter must be splinted in most cases using a thin catheter, because the stoma may narrow slightly and to impede the flow.

In dry stomata are the “collection bag for urine from the small or large bowel loops, they are in the interior of the body and must be emptied every 3-4 hours. For an ileal conduit, the ureters are implanted in a remote off small intestine content. This segment is sewn formed like a nipple in the abdominal wall. In a colon conduit, the ureters are implanted in a remote off colon portion. This proportion is also nipple formed sewn into the abdominal wall.

An alternative technique is to ileostomy continents to call the ileoanale Pouch (J, S, or W-pouch) and the Kocksche Case (Kock pouch). Not in every case is one of these surgical options available. When a ileoanal Pouch as an alternative to an ileostomy, after removal of the colon, the last small bowel loops, an artificial reservoir created similar to a pocket. This reservoir has an opening at the lower end that is connected to the sphincter apparatus. With intact sphincter, the chair collects in the pouch and can then be excreted as before the anus. In the hard running ulcerative colitis it is usually conceived as a J-Pouch by highly experienced surgeons.

In Kocks Chen pocket is also formed similarly from the lower small bowel loops, an artificial reservoir bag. Another piece of intestine is removed from the small intestine and used as a link from the Kocks's pocket to a small opening in the abdominal wall. Due to the surgical technique ensures that can reach during the filling phase of the reservoir no chair through the opening in the abdominal wall to the outside, first by introducing a catheter to empty the reservoir. Today ileoanal Pouch in the method of choice is preferred to the “Kock pouch”.

It is estimated that in the western world of 1000 people are living with a stoma.

Usually have stomata of the Small intestine (ileostomy) and Urostomata be derived in a stoma bag. The thin liquid intestinal contents in intestinal discharges, depending on food intake about 2,500 ml/day. The large intestine is off and meets its normal function (water and salt balance) is not more. To the patient means that he always wear a stoma bag to the body must, in the hour (emptied at the ileostomy), on average, about 80-100 ml of liquid. The amount of fluid can be regulated by adapting drinking and eating behavior, but may not fall below the daily mean certain quantities, to include disorders of the water and thus to avoid renal dysfunction (loss of fluid and electrolytes).

In addition to carcinogenic diseases of the colon can also inflammatory diseases of the colon and small intestine (eg Sigmavivertikulitis, Crohn's disease or ulcerative colitis) to the need of an small intestine stomata. Especially patients with diseases, including Crohn's disease or ulcerative colitis have often given a temporary ileostomy and often a definitive ileostomy. This is necessary for persistent fistula in the rectum area be brought to the healing or with poor sphincter function. Patients with inflammatory bowel disease are mainly young patients and should be the case involved many years living with an ileostomy.

The indication for the construction of a Urostomas (ileal conduit or “Bricker bladder”) and others given in the presence of bladder ureter tumors, atresia and birth defects, shrinkage bubbles (radiation damage), gynecological tumors, neurogenic bladder emptying disorders, tumors of the pelvis and injuries.

Irrigation is a term from Greek and means “irrigation” or “flush”. Such a purging of the intestine at regular intervals, causing it to regular emptying. After the irrigation for 24 to 48 hours no precipitation is expected, so that will be worn in this time, no bag, but only an inconspicuous Stoma cap. However, a stoma for irrigation only with fixed shaped precipitates suitable and even then only under certain conditions. For a special set of Irrigation is needed.

### STATE OF THE ART

For the protection of different stomata with specific activities of the industry a whole range of different fashionable Stoma-bandage or Stoma-bodice for men and women has developed. In these bandages and corsets usually is a bag for the bag and the patient should offer integrated security and freedom in leisure, sport and work.

The range of products for ostomy care is almost unmanageable size. Mostly the products they use one-piece provide care that is the skin plate is fixed to the bag. If the bag is changed, the skin protection must be removed. To avoid overloading the skin, one piece should be changed more than three times a day. One piece is very flat, with little wear, are flexible and easy to use.

The two supply includes a base plate for skin protection and an associated bag. An index ring on the base plate is attached, in which the bag can be simple connected. The base plate can remain for several days on the skin and the bag still be changed as often.

Types of bag are sealed bags, which are typically used for the colostomy, as the chair for some time after surgery is usually shaped back down. Closed bags usually have a charcoal filter that allows to escape the pressure resulting from the digestion gas smell. If required, the closed bag to be changed two to three times daily.

Open bags are mainly used in ileostomy, as there is frequent low to pulpy precipitates. ileostomy bags have a bottom outlet, which is closed with a clamp, so the person can empty the bag on the floor outlet and not constantly entire supply has to change. Most of these bags without an integrated filter, however, be offered by some manufacturers also open bags with an integrated filter.

Stoma with small intestine-/Urostomata are due to the high production volume of liquid forced a continuous supply of bags to carry.

A brief change to alternative Stoma-caps how they are used for irrigation in colostomy and thus a temporary “relief” from the bag-in is not for technical reasons.

Devices and equipment for a temporary stoma closure are known (eg from U.S. Pat. No. 6,050,982 and WO 90/07311). They are used for irrigation in colostomy, and thus represent a time-limited “liberation” of a stoma bag, for an ileo- or urostoma carrier but for technical reasons not possible. In an ileo- or urostoma involves large amounts of liquid for which a conventional colostoma-closure is neither suitable nor designed. For this purpose, so far only the known stoma bag use. The affected stoma must arrange their lives with the bag. Affected are exceedingly young patients suffering from inflammatory bowel disease. The sensitive skin around the stoma must be treated to gentle. This is especially true for people affected by ileostomy because the stool is very aggressive here. When skin is so absolute density, odor protection and skin-adhesive or adhesive substances to be respected.

### SUMMARY OF THE INVENTION

Object of the invention is to provide a cost effective and easy-to-use device that allows a person concerned in particular with ileostomy/urostoma and also colostoma—care to get along for some time without the need to supply bags.

According to a first aspect, the invention provides a device according to independent claim 1, equivalent to a device for a temporary stoma closure with a base plate for permanent connection to a body surface of a person, such as the abdominal wall and a cover that is permanently connected to the base plate for forming a temporary stoma closure and is so shaped to a leaking content. Recording volume is on the reception of a container for exiting from the stoma content.

According to an alternative aspect, the invention provides a device according to independent claim 35, d. is, a device for a temporary ileo- or urostoma or colostoma shutter, comprising a base plate for permanent mounting in the periphery of a stoma on the outside of the body surface, and a cover that is permanently connected to the base plate for forming a temporary stoma closure, and on the inside the cover into the stoma protrude.

Other aspects of the invention emerge from the dependent claims, the following description of embodiments and the accompanying drawing.

### BRIEF DESCRIPTION OF THE DRAWINGS

Further embodiments of the invention are now described by way of example with reference to the attached drawing. In the drawing is schematically shown:

FIG. 1 a terminal ileostoma;

FIG. 2 a double-ileostoma;

FIG. 3 a colon conduit;

FIG. 4 a Ileum conduit;

FIG. 5 a schematic sectional view of the inventive arrangement with a bottle cap and a cover plate on a base plate;

FIG. 6 a schematic representation of a device of the invention from above;

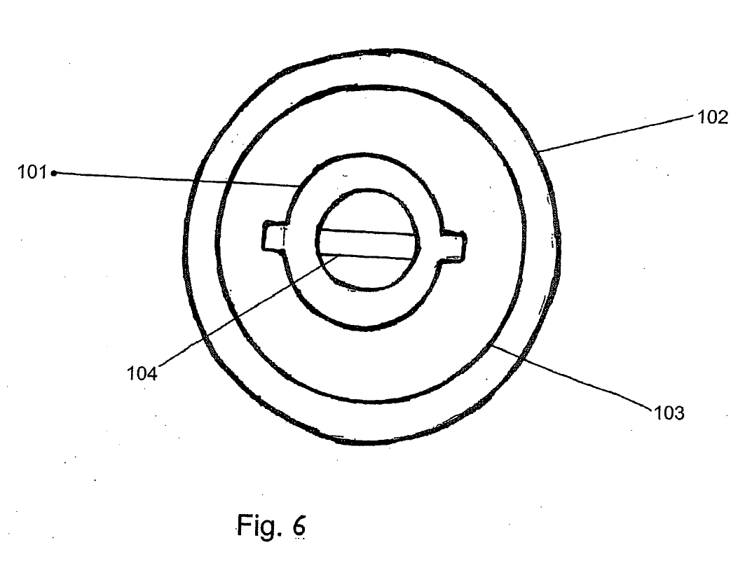

FIG. 7 a schematic sectional view of an inventive device with an outer and inner foil container;

FIG. 8 a schematic sectional view of an inventive device with the cover on the base plate with an insertion;

FIG. 9 a schematic sectional view of an inventive device with the cover on the base plate with a valve mechanism;

FIG. 10 a schematic sectional view of an inventive device with the cover on the base plate with an optional balloon can be filled;

FIG. 11 a schematic sectional view of an inventive device with the cover on the base plate with a shield;

FIG. 12 a schematic sectional view of an inventive device with the cover on the base plate with a split cylinder, and

FIG. 13 a schematic representation of the cylinder of FIG. 12. and

FIG. 14 a schematic representation of an alternative embodiment of a device for a temporary ileo- or urostoma-closure.

The figures are not necessarily to scale, some parts are represented only symbolically, attention was paid to the principle of the invention.

### DETAILED DESCRIPTION OF PREFERRED EMBODIMENTS

Before the description of embodiments of the inventive device, here, following some images of different types of stomata are explained for a better understanding.

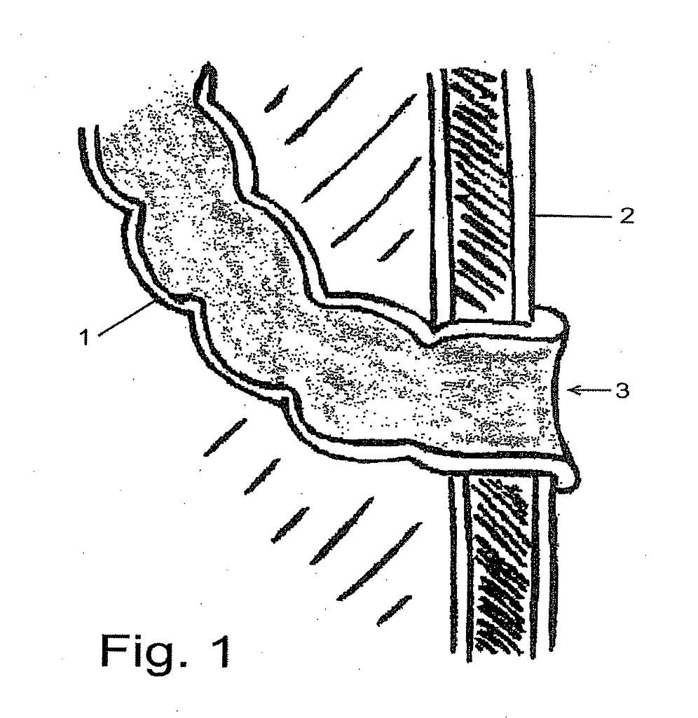

FIG. 1 shows a terminal stoma with an intestinal feeding, which is guided through the abdominal wall 2, and 3 is there a stoma.

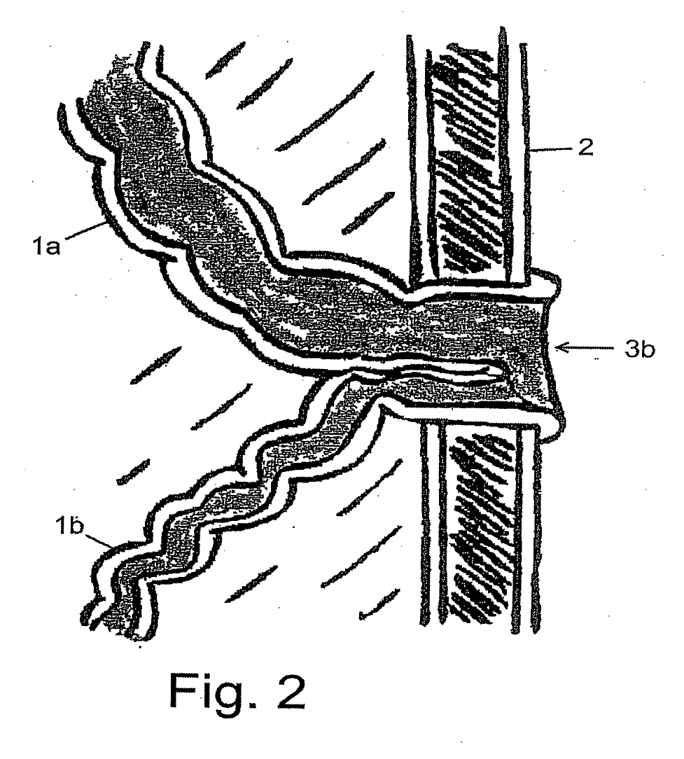

FIG. 2 points to a further embodiment of a double-ileostomy with a feeding intestinal Part 1a, 1b a laxative bowel part and the stoma 3 to the abdominal wall 2

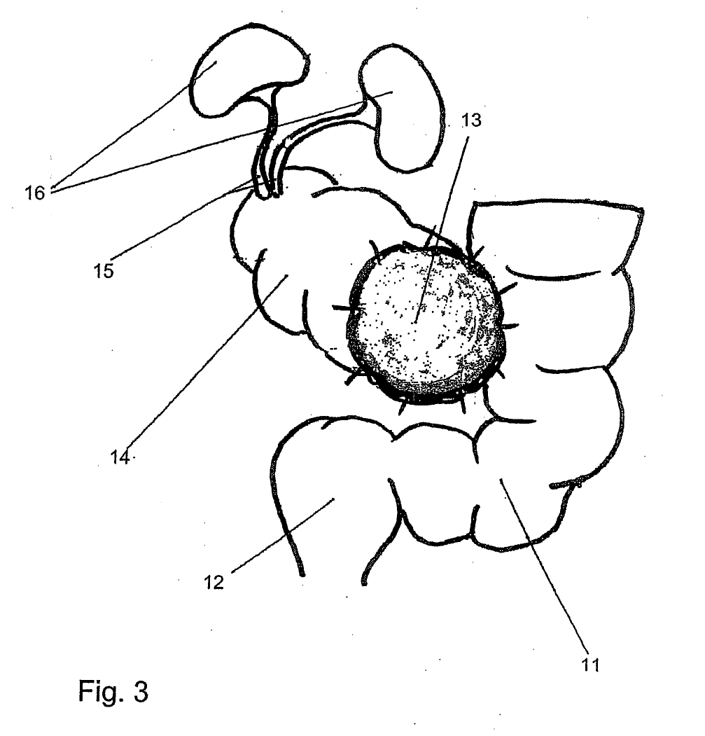

FIG. 3 points to a further embodiment, a colon conduit to be drained in the renal ureter implanted 16 on 15 in a switched off colon segment 14 The colon segment 14 opens into a stoma 13 which is sewn into the abdominal wall (not shown). In the background are a Colon (Sigma) 11 and see a rectum 12

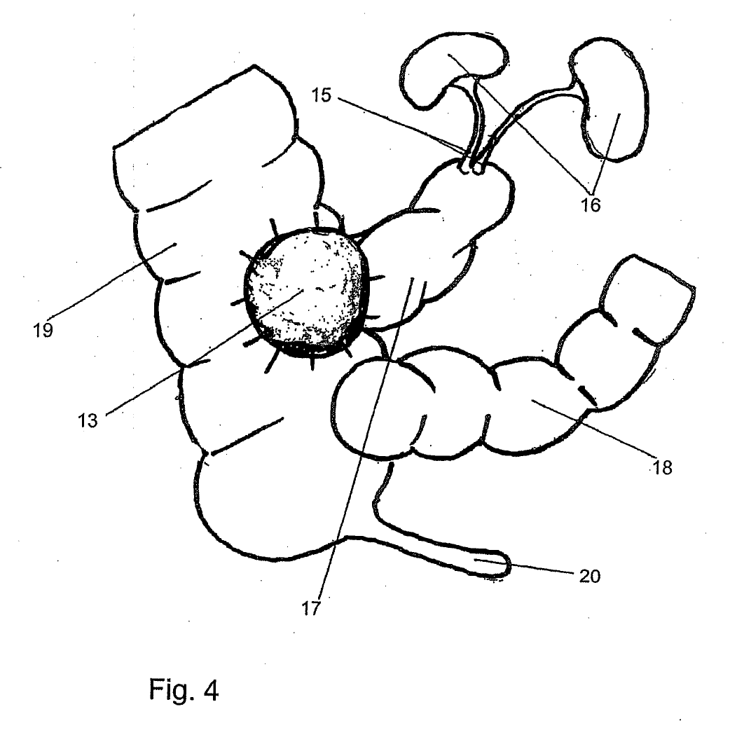

FIG. 4 points to a further embodiment, an Ileum conduit in which the kidneys are drained 16 on the implanted ureter 15 in a deactivated small intestine segment 17, whereby the small bowel segment ends 17 in a stoma 13th In the background a small intestine 18, 20, and an appendix a colon can be seen ascending 19th In the following, various embodiments of the inventive device for a temporary ileo- or urostoma-shutter are described in more detail. Here, the term “stoma shutter” on one or a ileostoma-shutter urostoma-shutter.

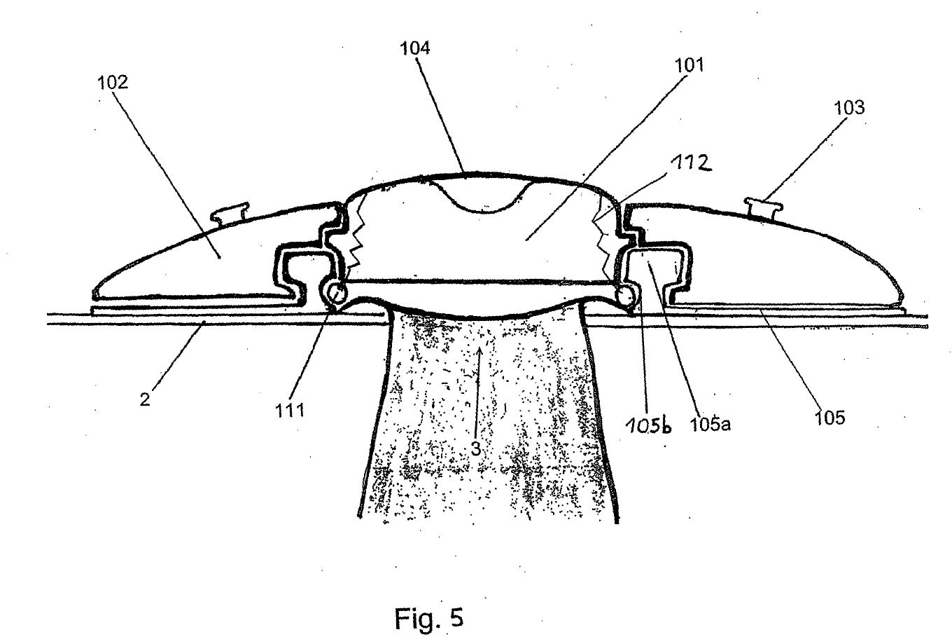

FIG. 5 shows a first embodiment of a device in a schematic sectional view. The inventive device is to replace one of the prior art bags temporarily. It includes a base plate 105 for this, which is designed for permanent installation in the periphery of a stoma on the outside of abdominal wall 2nd Furthermore, a cover detachably connected to the base plate 105 for forming a temporary stoma closure and in the manner of a half-shell closure capsule 101 so formed is that it is a record volume for receiving at least described a container in conjunction 112 with the following figures in more detail, for from 3 leaking fluid forms the stoma. As seen in FIG. 5, the container is in its open end detachably coupled to the base plate 105^{th}. The cap 101 can also be solved independently of the container from the base plate. The base plate 105 has a circumferential recess 105b provided to which the container can be mounted at its open end.

On the base plate 105 with an extensive index ring 105a, which is applied to the cleaned and degreased abdominal wall 2, flat cover plate 102 is attached. This cover plate 102 can be designed as a reusable unit and are coated with an anti-odor agents. The coating can with metal oxides, which are known to be good catalysts. This can to form nanoscale particles in a sol-gel integrated network and applied to the cover plate 102 In addition, a coating that will contribute stain (Lotus effect) are used. Variations of such coatings are already as “Anti-scratch-paint” known from the car industry. In the cover plate can be accommodated in one embodiment of an electronic receiver unit for a level sensor. The location of the receiving unit is designed so that a mechanical or electronic contact with the sensor, indicating the filling state of a stoma exited/Urostomas, is possible. The cover plate 102 has a central opening in the middle, the half-shell closure capsule 101 to use. The ventral surface of the cover plate 102 can have an index ring 103rd Remove the locking cap 101 can thus either a normal stoma bag will be attached. Preferably, the cover plate and the sealing cap from a non-deformable or only slightly deformable material such as plastic are formed. In a further embodiment form the panel and the closure cap an integral unit.

The inventive cover 6 shows an embodiment of a device with top 102 in the top view. The interchangeable inner closure cap 101 can serve as a replacement bag. The closure capsule 101 is mounted for example by a bayonet closure, with a lowering and rotation in the cover plate 102^{nd}. This is on the closure capsule intervention 104^{th}. In an alternative embodiment, cover plate and closure cap be constructed in one piece.

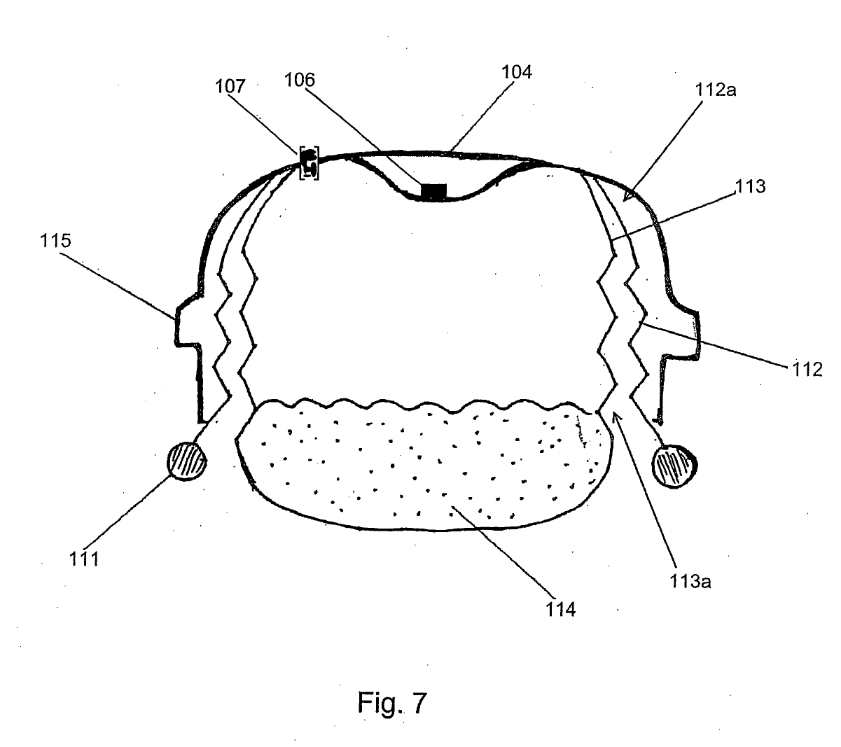

According to FIG. 7, the closure capsule is designed to be 101 according to one embodiment of the invention as Plastic half shell coverage is included in the interior of a folded accordion container foil 112. The container foil 112 forms a first film tube 112a. The first film tube unfolded after the shutter release capsule 101 and can accommodate in this way after removing the cover, an intestinal contents. The three facing the stoma input of the first film tube 112a has a circular, circumferential bead ring 111, exit of the Stoma in the skin level in a recess at the base plate 105 or cover 102 bears and seals. In one embodiment of the first film tube is produced in whole or in part from a metallic material. After filling the film tube 112a is to be achieved on the closure capsule through a rotating motion that the outlet of the film tube by the rotation closes and the contents can not leak out. After filling of the film tube rotating movement causes due to the short-term liability of the bead ring 111 to the base plate 105 a twist of the film tube. This attachment causes a rotational movement of the hose, that the metal portion of the tube closes rotating. Then, the foil containers are filled from the base plate. Alternatively or in addition to securing the contents of one can be placed in the market on one-time-use clips.

The closure capsule may be a filter 107, shall include an activated carbon filter, which allows the pressure to escape resulting from the digestion gas smell.

In a further embodiment of the invention is in the closure capsule 101, a second folded foil container 113, which is permeable for example, through small holes, for fluid. This second film containers can be secured to an end at the plastic half shell and will be included in the first film tube of the first container foil 112. She moves like a doll in the doll. In one embodiment, the foil container 113 slightly shorter than the outer film tube. The hoses are not in your basic form of limited and may have other than round cross sections. Before the release of the closure capsule 101 from the cover of the container foil 113 forms a folded foil inlay. The container foil 113 forms a second film tube 113a and includes a source material 114, preferably made of organic material, which expands at a low volume of their own after contact with liquid and can absorb several times its volume. Such swelling agents are also known as a super absorber and incontinence articles are well known. Doing so, the perforated foil container with the source as a waterproofing agent 114 in the plug stretch exit of the Stoma and thus absorb more liquid. Alternatively, the second film tube 113a of the first film tube 112a may be replaced by a liquid and/or elastic film container subdivided which separates a region with swelling agents 114.

These measures are designed, first, that the exit of the Stoma is sealed, the second is through the liquid recording the pressure on the cap, thereby increasing the density. To view the need for a cap change, the level of film content via pressure or pH or conductivity of the source agent 114 by a sensor 106 is covered. Other possibilities are volume measurements, strain gauges of film tension and voltage. The measured value may on wireless technology such as for example RFID, ZigBee or WLAN passed on to appropriate recipients and there (mechanical alarm clock, vibrator) or optical (LED lights) to the institution will be displayed. The signal can be designed depending on the urgency differently. To fasten the cover plate, the closure capsule includes a projection 115.

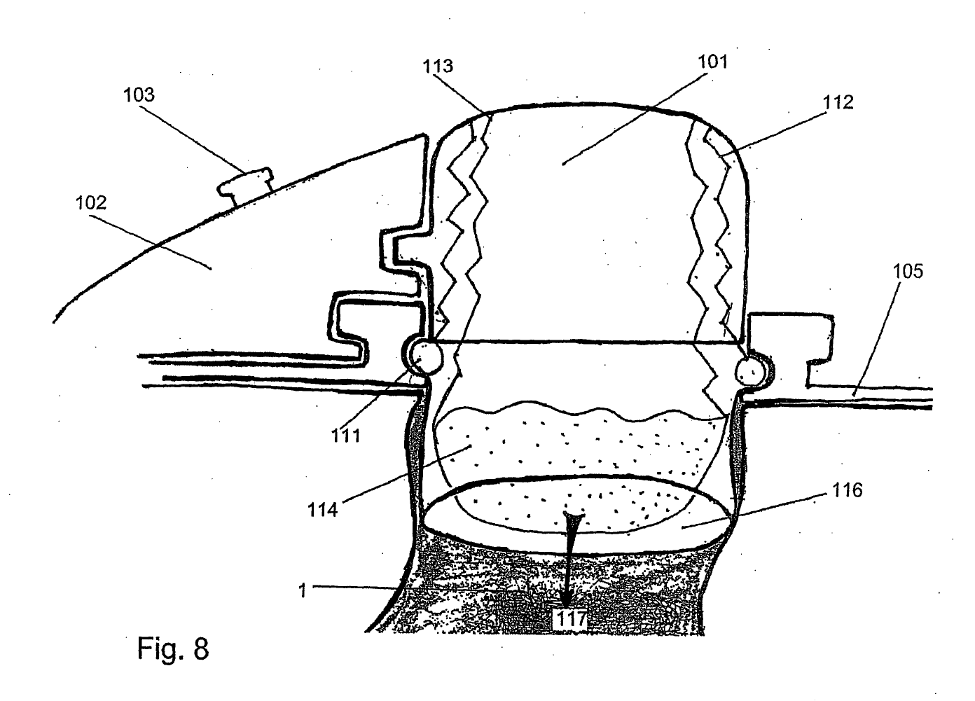

In one embodiment, as shown in FIG. 8, the invention comprises an insertion in the la afferent intestine. This includes an introducer tube 116, for example, a soft silicone tube in the form of a cylinder which is fastened at one end to the base plate. This tube serves as a guide for the inner film tube with the organic swelling agent 114. The inner container foil 113 with the source means 114 moves in through the tube 116 in the intestine. The direction is indicated by an arrow 117. The tube is designed to prevent the film tube is jammed in front of the stoma and increased by the volume increase of the closure capsule 101 with the base plate 105, which could lead to a leak on the exit of the stoma.

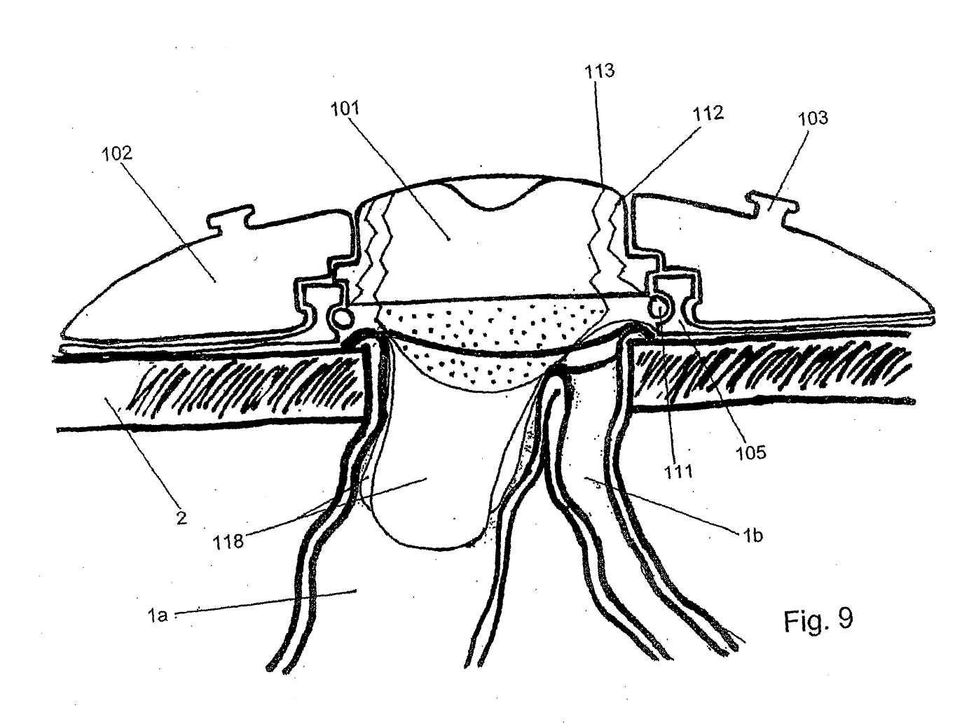

FIG. 9 shows a further embodiment with a ring-shaped soft plastic sail 118, which is similar to the valve at the terminal ileum and caecum transition into the stoma. This flap is shaped so that it results in a very effective closure in a retrograde direction in capsule change. During stretching of the foil container with the source means 114, the plastic sail is 118 in expanded passively into the intestine. After removing the foil container is filled the soft plastic as a sail Mucosal fold together and inhibits the uncontrolled leakage of liquid from the stoma. Attaches the annular plastic sail to the base plate 105 is directly on the stoma.

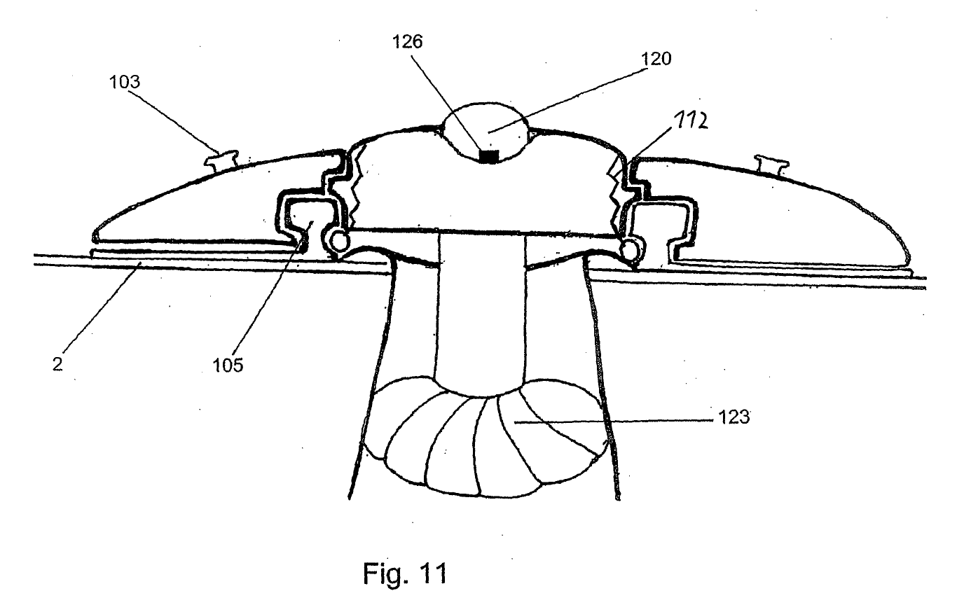

In an alternative embodiment, as shown in FIG. 11, the device comprises a connector, eg a tube 120, preferably made of silicone, which is fastened with one end in the middle of the capsule inside. At the other end of the tube—in the intestinal lumen located—is a container foil 123 in the form of an umbrella attached unfolding triggered by the onset of the closure capsule as a curved screen, by the liquid pressure from the intestinal lumen. Here, the screen is nestled on the side wall of the intestinal lumen and causes some fluid retention. By means of a sensor 126 leading to the fortified in the closed capsule tube has 120 contact can be measured as the fluid pressure in the intestinal lumen and are passed to those described above ads. Liquid, which pushed past on the outside edge of the screen is determined by a swelling agent, as in the example of FIGS. 7 and 8, absorbed. After exceeding a certain pressure ratio (volume-), which is predefined in the measured values of the sensor, which is rotated in the closed capsule suspended silicone tube, for example, 180°, and the foil shield does 123 like an umbrella. The increase occurring liquid that flows subsequent, is absorbed from the source material. The closure capsule 101, is housed in, as in the above examples, a folded container foil 112, with silicone tube is removed in the manner described above. The closure is to prevent the leakage of fluid through the above-described soft plastic sail 118.

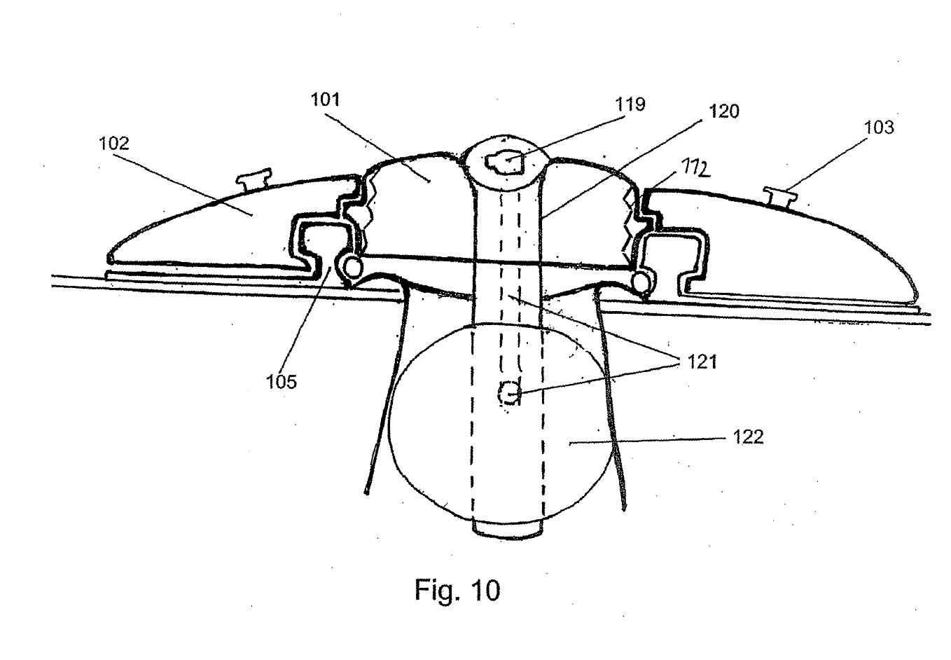

According to FIG. 10, an embodiment of the invention, include, instead of the foil shield, which unfolds to an extension tube, an inflatable balloon 122 that is attached to the tube 120 and according to the principle in urinary catheter balloon obstruction, intubation tube, etc. works. This balloon is inflated via a channel 121 in tube 120 and hugs as balloon occlusion at the inside of the intestinal lumen. Balloon edge at the push past liquid is absorbed in the example of FIG. 7 or 8, the source of funds 114. After exceeding the pressure ratio, as described above by a sensor 119, which came to the fortified in the cap tube contact and measures the fact exerted pressure, displayed, must be vented before removing the closure cap 101 of the balloon 122. This is done by opening a lock, for example in the form of a cap or a valve (not shown), at the capsule exterior. The balloon collapses, resulting liquid is absorbed by the swelling agent 114. Balloon and bottle cap with the slide content may be removed. The plastic sail 118, which serve as a flap to prevent a further leakage of fluid.

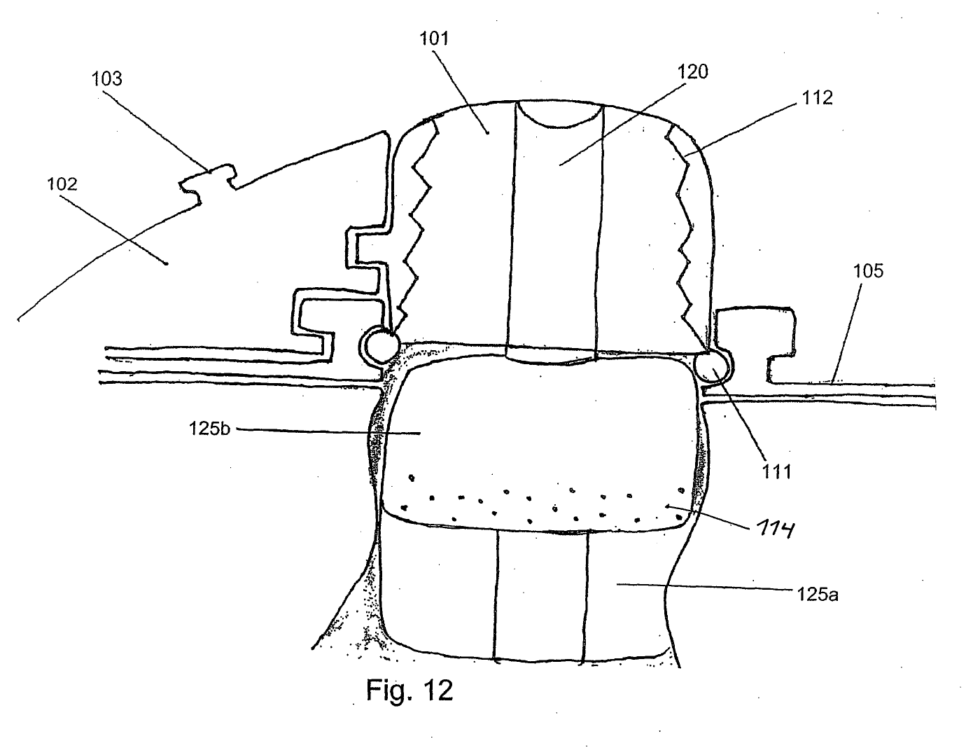

In a further embodiment of the invention according to FIG. 12 in place of a balloon one cylinder is provided, which in the same manner as described above for balloon, is inserted into the intestine through a feeding arranged on the cap 101 extending 120. The cylinder has two chambers. The lower chamber 125a directed to the gut lumen serves as the introducer and the balloon 122 in FIG. 10 inflatable. The upper chamber 125b is the inclusion of the intestinal contents (similar to the inner foil containers 113 in the example of FIG. 7) and is filled with a swelling agent 114. To empty the cap 101, where as in the above examples, a folded container foil is accommodated, with the cylinder as described above, removed, so that the cylinders are disposed of together with swelling agent in the container slide.

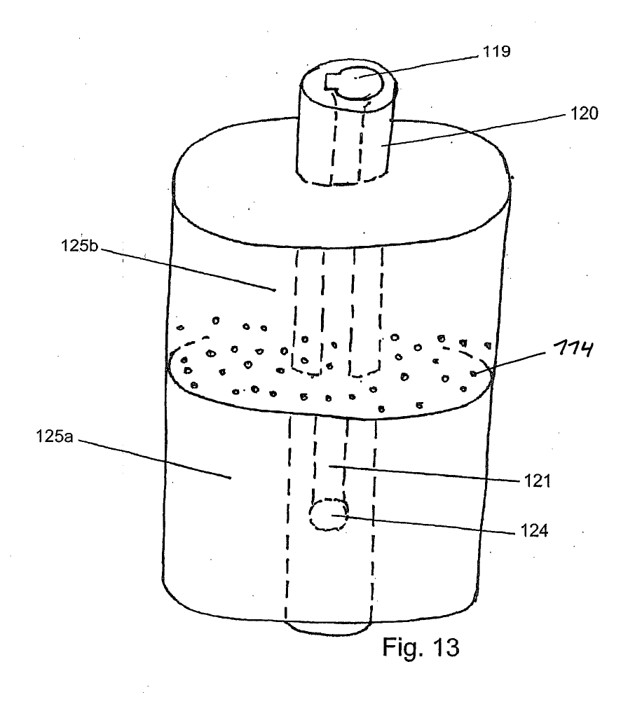

FIG. 13 shows the cylinder is presented in detail. As in the embodiment with a balloon (see FIG. 10) of the cylinder through a tube 120 attached to the cap through which a channel for ventilation in FIG. 13. The cylinder is presented in detail. As in the embodiment with a balloon (see FIG. 10) of the cylinder through a tube 120 attached to the cap through which a circuit has led to the ventilation. The tube 120 has in the lower chamber 125a of opening 124 to the channel. At the upper end of the tube 120 is a ventilation opening 119. Due to the cylindrical design, the introduction to the stoma is considerably easier.

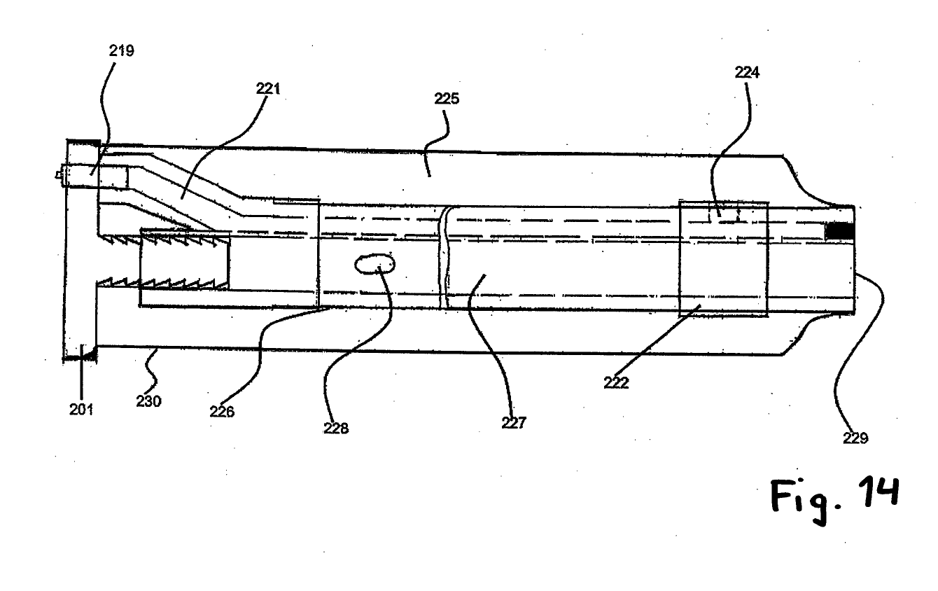

FIG. 14 shows an alternative embodiment of a device for temporary ileo- or urostoma shutter. The length of the closure unit is the specific application area, ie whether ileo or urostoma, adjusted. The device includes in the stoma protrudes on one side open tube element, which serves as an insertion and provided on the to the gut lumen directed end portion an inflatable volume, such that the tubular element is fixed in the intestine and seals simultaneously through the inflatable volume of the stoma. Furthermore, the device comprises a chamber that is arranged around the pipe, and designed to accommodate the intestinal contents.

In a preferred embodiment as a modified catheter tube element 226 as soft silicone used which is equipped at the top with an inflatable balloon 222 made of silicone. Such so-called balloon catheters are typically used for transurethral bladder drainage. A balloon catheter is usually made of soft silicone or latex and is near the top provided with a small inflatable balloon made of silicone, which, if it is inflated, the balloon catheter normally blocked in the bladder.

The catheter used in the present embodiment 226 has a diameter of about 28-30 Charriere (about 9-10 mm). An axially extending main channel 227 of the catheter 226 connects an input port 229 with an opening 228 to a chamber 225, which is designed as an elastic foil container 230 of cylindrical shape and is arranged around the catheter and trained around 226 for receiving the intestinal contents. The catheter 226 is held at one end to the inside of a cap 201. The entrance opening 229 at the other end of the catheter 226 opens into the intestinal lumen. The edge of the inlet opening 229 is rounded and soft. The chamber 225 is arranged around the catheter 226 and is from the inside of the cap 201 at the stoma and limited by an elastic container from the sides. In the illustrated embodiment, the container is formed by an elastic film container 230 in cylinder form. That the distinction is the inner lumen is the intestine, and contains a swelling agent (not shown) as already described in connection with other embodiments. In a specific embodiment of the main channel 224 also contains a swelling agent.

The flexible foil container 230 is preferably made of a tube made of thin silicone or latex, which coats the catheter 226 and the chamber 225 The flexible foil container 230 is distal to the balloon 222 in a ring near the Entrance opening 229 with the catheter 226 connected, for example, glued or welded, runs over the inflatable balloon and the proximal end to the inside of the cap 201 as associated with an explosive or press ring. This is done in the inside of the cap 201 is a recess or countersunk circular groove into which the proximal elastic foil containers can be securely anchored by a clamping connection. The foil containers 230 thus forms a closed system to the sides. The foil wrapped container 230 while the catheter and the distal end of the inflatable balloon 222 and is connected to the catheter 226 and the main channel 229 and sealed at the proximal end in the end cap 201, anchored. The foil container 230 is sized so that it will release into the stoma 3 loosely placed around the catheter 226, and is designed to appeal to the filling of the chamber 225 with fluid from the Intestine to stretch around the cylindrical inserted catheter 226 to the intestinal wall, out. The maximum achievable diameter of the foil container 230 in the gut is influenced by the intestinal wall elasticity.

In this embodiment, the oval opening 228 and located near the cap of two hundred and first The opening 228 in the main channel 227 is in one embodiment with a membrane (not shown) is provided, which flow as a one-way opening such as a check valve only liquid from the main channel 227 in the chamber 225, can. The membrane is sealed with a change in body position of the carrier opening 228 between the chamber 225 and the main channel 227, and prevent a backflow from the chamber 225 in the main channel 227.

Inside the balloon catheter 226 connects channel 221 to a port 219 of the 222 cap 201 with the balloon The balloon channel 221 flows into the balloon 222 in an opening 224 in the wall of the balloon channel 221. About the port 219 and the balloon channel 221, the balloon 222 with the help of an injection device, such as a be injection syringe with air or a liquid filled, for example, with a volume of about 5-10 ml. In this way, the catheter is kept in the intestine 226 and seals it in place of the expanding balloon 222 to the stoma 3 from three down. The port 219 is provided in some embodiments with a check valve, which is running pilot operated in specific embodiments. The check valve holds the balloon 222 inflates, while the catheter 226 to remain in the intestine. If the check valve unlocked at port 219, for example, by depressing a push button in the form of a running tool, the filling of the balloon 222 released and the catheter is held not by the balloon 222nd The balloon channel 221 is closed near the entrance opening 229.

To use this device of the invention is not provided on a catheter 226 in the stoma. When the catheter 226 is inserted far enough, the carrier is the cap 201 in the cover plate 102 and locked them there. After blowing the institution the balloon 222 e.g. with a syringe. The inflated balloon 222 holds the catheter 226 prevents the intestine and intestinal fluid that flows past the outside of the catheter.

As long as the catheter 226 is inserted in this way, intestinal fluid passes through the entrance opening 229 from the intestine into the main channel 227 of the catheter 226. The intestinal fluid then flows through the opening 228 from the main channel 227 in the chamber 225, which is surrounded by the foil container 230. A swelling agent in a plastic container 230 takes the intestinal fluid on the foil container and 230 widens. In one example, the swelling agent binds the intestinal fluid in the chamber 225 in jelly-like form. Once absorbed in the intestinal fluid chamber 225 remains permanently attached, which also simplifies the later disposal. The funding source also supports a capillary due to the absorption of intestinal fluid in the chamber 225 and the extension of the foil container 230 also, which can be absorbed more liquid in the chamber 225. It is also absorbed by the capillary effect of the swelling agent even in a small intestinal pressure, intestinal fluid in the chamber 225.

In a further embodiment of the cap 201a flush port (not shown) is arranged, which is also accompanied by a simple non-return valve and from which extends an irrigation channel in the direction of the main channel 227. The irrigation channel flows between the cap 201 and the 227 opening 228 in the main channel About the irrigation channel, a rinsing liquid such as water or a saline solution injected with a syringe if needed in the main channel 227. The fluid is to keep the main channel 227 permeable, in particular, they should remove any adhesions with fibrous material, protein flakes, etc. These solid components are introduced with the NaCl solution and washed into the intestinal lumen. In a specific embodiment, the opening 228 is closed before the irrigation fluid is injected into the main channel 227. This example closes a valve opening 228 when the syringe is inserted into the irrigation port. This will prevent the irrigation fluid escape during flushing through the opening 228 in the chamber 225.

In a further embodiment is a sensor for measuring the level on the catheter 226 or the inside of the chamber 225, provided that an appropriate signal to a receiver as in the cover plate 102, before the foil container is full, sends. The sensor is for example a pressure sensor that is triggered by the rising funding source, or another of the above mentioned sensors. The carrier then receives as described above, the measured value or a recommendation to change the cap 201 with the catheter 226 and the foil container 230 and its contents.

To remove the cap 201 with the attached foil container 230 and its contents is first deflated the balloon 222 on connecting 219. Including bringing the check valve will unlock through a syringe, or by inserting the above-mentioned push button that unlocks the valve. In one embodiment, the carrier releases the cap 201 from the cover plate 102 and pulls the foil container 230. The swelling agent in a plastic container will hold the liquid inside the film container 230. A container foil, container foil, such as the 112 from FIG. 7, which is housed in the cap 101 and is held in the cover plate 102 can be provided in addition and wrap the foil container 230 during extraction.

In another embodiment, such a foil container (not shown) until just before removal of the catheter 226 attached. To a separate container foil (like an accordeon) folded foil container is constructed. After the balloon was deflated 222, the foil container is clamped with an open end on the index ring 103 of the cover plate 102. The container foil is designed so that the engagement of the carrier 104 to the cap 201 can be taken by the container slide. For this purpose the container foil is either thin and pliable, or even run with a hole provided through which the cap 201 can be taken two hundred and first If so, the edge of the hole are completely glued to the cap, so that no escapes intestinal fluid. The carrier opens the closure of the cap 201 about the operation 104 and pulls the catheter 226 with the foil container from the interior of the bowels. The container foil wrapped the cap 201 and the foil container 230 with its contents in full. The container foil thus allows a non-contact and safe disposal.

After removal of the contents, the stoma is open, and provides only three short periods with the cover 102. The user can now either place another closure cap 201 or muster with another catheter 226 a stoma bag on the index ring of the cover plate 102.

As with a stoma bag should change when changing the cap 201 is not much liquid bowel contents leak from the stoma. When changing stoma bags of sufficient normally a short recording of fluid by swabbing the stoma 3 with a compress.

A further non-illustrated embodiment of the invention includes at least one, preferably two half-discs, which are spring-mounted and fixed to the upper edge of the base plate. When inserting the closure cap in the cover plate, the two soft Half discs approximately parallel to the abdominal wall in each of the corresponding half of the cavity of the cover plate back. Through the simple spring mechanism, these two Half discs removal of the closure capsule and compressed form over the exit of the Stoma a kind of valve lock. This serves as an additional safeguard against leakage of intestinal contents when changing the capsule.

The embodiments of the inventive device provide practical tools for patients with an ileostomy or urostoma, in which the ureter via a conduit ileum or colon conduit be expelled. The teachings of these embodiments can be in principle but extend it to all stomata, affecting the digestive tract. It is intended as a limited period, the waiver of a conventional stoma bag allow. An artificial bowel or bladder outlet greatly influenced the social—as well as the intimate lives of those affected. Because of the taboo subjects of urine and stool frequently do shame and inferiority. The invention provides a temporary cover of a stoma, which is equipped so that was added when changing the cover leaking fluid in a container or is introduced into the intestinal lumen, there is already recording fluid in a container. A relief in dealing with these drastic changes to be achieved with the embodiments of the inventive device, raising self-esteem and quality of life and mobility of the affected be improved. Next create the embodiments of the invention is an inexpensive and easy to-use device that enables a person concerned as with ileostomy/urostoma/colostoma supply allowed to get on for some time without the need to supply bags. Likewise, the embodiments of the invention is to provide a safe stoma care, stoma to comment on the “privacy policies on the different” to give people a new self-esteem and make it more socially acceptable.

1. Device for a temporary seal of a stoma comprising:
a base plate (105) for permanent application in the periphery of a stoma on the outside of a body (2) and a cover (101, 102) which is connected releasable to the base plate (105) for forming a temporary stoma steal and is configurated in such a way that it forms a volume for receiving at least one receptacle for fluid emerging from the stoma (3),
wherein the container is detachably coupled with the base plate (105) and the cover (101, 102) can be solved separately from the container from the base plate (105),
the base plate (105) comprises a recess (105b), in which the container can be mounted at its open end,
the cover is formed of a closure capsule (101) and a cover plate (102), which the closure cap (101) in an opening of the cover plate to the base plate (105) over the opening of a stoma (3) couples,
the cover plate (102) includes an index ring (103) surrounding the opening of the cover plate to a stoma bag in the periphery of a stoma (3) to fluids and releasably to connect with the cover plate,
the closure capsule (101) is attached by means of a bayonet-lock in the cover plate (102), and
the container is designed like a tube (112a) which is open at one end.

2-9. (canceled)

10. Device according to claim 1, wherein the tube (112a) is formed like a foldable material and housed under an accordeon-like folding in the host volume of coverage, and the tube (112a) is at least partially formed from a metal foil container (112).

11. (canceled)

12. Device according to claim 1, wherein the tube (112a) at the open end of a bead ring (111), which is designed to in a circumferential recess on the base plate (105) and/or cover (102) was added to are and seal the opening of the base plate to the outside.

13. (canceled)

14. Device according to claim 1, wherein the tube (112a) is arranged in the closure cap (101), attached with the closed end of the closure capsule and is designed is to appeal to the removal of the closure cap from the cover plate (102) to unfold and out of the stoma (3) emerging intestinal contents to be incorporated.

15. (canceled)

16. Device according to claim 1, wherein the tube (112a) comprises a liquid-permeable wall (113) which establishes an area for absorbing liquid, the region for accommodating fluid is formed as a further tube (113a) and at least partially deformable and/or is designed foldable, and the further tube (113a) is shorter than the tube (112a) and is attached at one end to the cover.

17-18. (canceled)

19. Device according to claim 1, wherein a swelling agent (114) is arranged in the tube (112a).

20. (canceled)

21. A sensor unit (106) for the device according to claim 1, which is designed to display because of a sensor value with the need for a cap change of the device.

22. A sensor unit according to claim 21, wherein the sensor unit (106) is designed to detect pressure, pH and/or conductance of the swelling agent (114), and an evaluation has to determine the need for a cap change.

23. Device according to claim 21, wherein the sensor unit (106) is configured to indicate the need for a change of a capsule base, by mechanical and/or optical signal.

24-26. (canceled)

27. Device according to claim 1, wherein attached to the base plate (105) a valve in the form of a plastic sail (118), that is designed to be by the spring means (114) behind the wall (113) opened in fluid intake and close the stoma after removal of the cap with foil—and fluid content.

28. Device according to claim 1, wherein the cover is a connector (120) with an umbrella-like element (123) connected, which is itself in the intestine and a way to get on the cover the opposite end of the connector by intestinal pressure or by exposure to develop a carrier and to inhibit fluid leakage, the connector (120) is configured to guide the intestinal pressure to a sensor unit (126) on the connector or in the cover, and the device is equipped to rise to the umbrella-element (123) from a specified internal pressure or intestines through the carrier to fold.

29-30. (canceled)

31. Device according to claim 1, the device comprising an inflatable balloon inserted into the stoma covers, fitted to be ventilated from the outside to seal the stoma (3) optional.

32. Device according to claim 1, being provided on the inside of the cover (101, 102) one protrudes into the urostoma cylindrical container, is the one to the gut lumen directed first chamber (125a), which is designed inflatable, and one adjacent to the first chamber second chamber (125b) which contains a swelling agent (114) for receiving the out of the urostoma (3) emerging content.

33. Device according to claim 1, wherein the cover and/or the base plate (102) at least partially coated with a material that counteracts an adhesion of dirt particles and/or od

34. Device according to claim 1, wherein the cover plate (102) or the base plate (105) at least one disc, which must be preloaded and configured the opening under the action of the sealed spring force over the stoma, if there is no cap (101) added.

35. Device for a temporary stoma shutter (ileo-Urostoma-colostoma), comprising:
a base-plate (105) for permanent installation in the periphery of a stoma on the outside surface of the body (2), and
a cover (201), which is solvable with the base plate (105) for forming a temporary stoma closure, wherein the inside of the cover (201) protrudes into the stoma tube element (206) is attached, and being formed around the pipe element (226) around a chamber (225), which is designed to accommodate a main axial channel (227) protruding into the tubular element (226) emergent content of the stoma,
wherein the main channel (227) is provided with an input opening (229) which is configurated to receive intestinal fluid, the input opening (229) is connected with an opening (226) of the chamber (225), which is porous so that the receipt intestinal fluid is permeable only in the direction of the main channel (227) into the chamber (225),
the chamber (225) and/or the main channel is filled with a swelling agent for taking up,
the chamber (225) is built by an elastic container (230), which surrounds the pipe element (226), is formed,
the tubular element (226) includes a balloon (222), the balloon is inflatable with the tube element (226) in the stoma (3) to fix and/or to seal the stoma (3) to the outside, and
the balloon (222) is inflatable and/or ventilatable through the cap (201) via tube element (226) in the balloon channel (221) and/or be discharged.

36-40. (canceled)

41. Device according to claim 35, characterized in that the balloon is blowing up and remain filled via a check valve.

42-43. (canceled)

44. Device according to claim 35, characterized in that the tube element (226) is formed as an catheter an integrated inflatable balloon (222) on the top of the catheter.

45. Device according to claim 35, characterized in that the device comprises a sensor unit to notify the necessity of a change on the grounds of sensor value.

46. Device according to claim 35, characterized in that a sensor unit is set up to print Pressure, pH value and/or to record conductance of the source means, and a signal conditioning-unit for determining the need of a change, and the sensor unit is designed to notify the need for a change with a mechanical and/or optical signal.

47-48. (canceled)

49. Device according to claim 35, further comprising a container foil comprehensive, which is designed, mounted on a recess of the base plate to be to allow a loosening of the cap (201) and the device when removed from the stoma to be incorporated in itself.
